(19) United States
(12) Patent Application Publication
(10) Pub. No.: US 2013/0288227 A1
(43) Pub. Date: Oct. 31, 2013

(54) METHODS AND COMPOSITIONS FOR CELL SEPARATION OF BLOOD TISSUES

(71) Applicant: Daniel P. Collins, Lino Lakes, MN (US)

(72) Inventor: Daniel P. Collins, Lino Lakes, MN (US)

(21) Appl. No.: 13/882,922

(22) PCT Filed: Oct. 31, 2011
(86) PCT No.: PCT/US11/58580

§ 371 Date: Jul. 12, 2013

Related U.S. Application Data
(60) Provisional application No. 61/408,862, filed on Nov. 1, 2010.
(60) Provisional application No. 61/408,862, filed on Nov. 1, 2010.

(51) Int. Cl. C12N 5/078

(52) U.S. Cl. CPC C12N 5/0644; C12N 5/0634; C12N 5/0641
USPC 435/2

(57) ABSTRACT

This document provides compositions and methods for cell separation. These reagents and techniques specifically agglutinate cells via surface antigen recognition and can be used to recover rare cell types in high yield.

### CROSS-REFERENCE TO RELATED APPLICATION

This application claims the benefit of U.S. Provisional Application No. 61/408,862, filed Nov. 1, 2010. The content of the foregoing application is hereby incorporated by reference in its entirety.

### TECHNICAL FIELD

The present invention relates generally to compositions and methods for separating cells. More particularly, the invention pertains to reagents that specifically aggregate erythrocytes and mature myeloid cells via surface antigen recognition, and stimulate homo- and heterophilic cellular adherence.

### BACKGROUND

Isolation of cells for in vitro studies or for applications in cellular therapies usually incorporates an initial separation of blood cell components mainly based on the bulk depletion of erythrocytes, which comprise >99% of the cellular mass of blood and other cell types which either provide no therapeutic potential (granulocytes) or contribute to pathology, or, in general, interfere with the ability to monitor the cell population of interest. Depletion of T-lymphocytes from bone marrow donations prior to implantation is a common technique used to reduce the incidence or degree of graft versus host disease, which is mediated by T-cells. The techniques used to deplete these cell populations differ depending upon the cell population that is to be removed.

Techniques used for erythrocyte removal are based on hypotonic lysis of erythrocytes, density gradient separation, or enhanced centrifugal sedimentation using heta starch. Hypotonic lysis, while useful in low volume in vitro studies, is inefficient and impractical for the large volumes of blood tissues processed for cellular therapies. If utilized in cell therapy procedures, erythrocyte hypotonic lysis is usually done as a final clean-up step to remove the final remaining erythrocytes that may contaminate a sample after bulk depletions by other methods.

Density-gradient separation relies on small differences in the density of different cell populations causing them to segregate at different levels in a fluid medium of variable density. However, the differences in density between the cell types are so small and the individual cells types are quite heterogeneous in size and density, the different cell subpopulations often get distributed throughout the medium instead of segregating to a discrete level in the density medium. This property of the cells in the density medium results in poor recoveries of cells and contamination with undesired cell types. In procedures that enrich for rare blood cell types such as hematopoietic progenitor cells, density gradient sedimentation generally results in poor recoveries. For example, the use of conventional density gradient methods to isolate progenitor cells such as CD34+ hematopoietic stem cells from umbilical cord blood results in a significant loss of the desired cells. See e.g., Wagner, Am J Ped Hematol Oncol 15:169 (1993). Use of density-based cell separation medium to isolate lymphocytes resulted in selective loss of different lymphocyte subsets. See e.g., Collins, J Immunol Methods 243:125 (2000). These separation methods have an addition contraindication for use in cellular therapies in that the chemical entities in the separation medium are often toxic if infused with the cells into the recipient, and additional steps must be performed to ensure their complete removal prior to infusion. Instrument methodologies such as elutriation also depend upon differential separation of blood components by density and suffer from similar deficiencies in performance.

An addition method used to de-bulk erythrocytes from blood cell units is the use of heta starch. This method is currently used by many blood centers to process umbilical cord blood. The blood cell unit is mixed with heta starch and then subjected to centrifugation. Heta starch, a non-toxic substance developed clinically as a blood plasma expander, stimulates the formation of erythrocyte aggregates that will sediment more rapidly than leukocyte components when sedimented at 50×g in a centrifuge. While this method is non-toxic and safe for the recipient, its performance in the recovery of important cell types (e.g., hematopoietic stem cells) is highly variable depending upon factors like temperature, age of sample (post-collection) prior to processing, cellularity (i.e., concentration of cells per unit volume) of sample, volume of sample, and ratio of anti-coagulant to blood sample. These factors can often result in the poor recovery of stem cells and diminution of the engraftment potential of the cord blood cells, increasing the risk for transplant failure. With the advent of cellular therapeutics such as bone marrow transplant, stem cell-based gene therapy, and immune cell therapy the success of these treatments is directly related to the actual number of the cells being transplanted. High yield recovery of these rare cell types from donor tissue could vastly improve the success rate of the transplant or immune therapy.

### SUMMARY

The invention provides efficient, non-density based methods for separating and recovering therapeutically valuable cells from peripheral blood, umbilical cord blood, bone marrow, or other blood cell containing sample. In particular, this invention provides an efficient method to specifically remove undesired cellular subsets that either interfere with monitoring cells of interest in in vitro studies or contribute to the development of pathology when implanted. The invention features compositions that fractionate blood samples by specifically aggregating erythrocytic and mature myeloid cells via surface antigen recognition and stimulated homo- and heterophilic adhesion molecule mediated aggregation, mediating the enhanced sedimentation of those aggregated cells at 1×g. The non-aggregated supernatant fraction is enriched for stem and progenitor cells and depleted in erythrocytic and granulocytic cells. Cell populations also can be recovered from the aggregate phase of the fractionated blood. Using these compositions, even very rare cell types can be recovered in relatively high yield. Cells isolated from either the supernatant or agglutinate have not been biologically modified by interactions with the components of this composition. The compositions and methods described herein can be used to prepare desired cells in high yield for tissue culture, immunophenotypic characterization, further purification, therapeutic administration, or other diagnostic testing.

In one aspect, this document features a composition that includes dextran; anti-CD15 antibody; heparin; and serum albumin. The composition further can include phosphate buffered saline. The pH of the composition can be between 6.8 and 7.8 (e.g., 7.2 to 7.4). The serum albumin can be bovine serum albumin or human serum albumin. The concentration of serum albumin can be about 0.5% to about 5%. The anti-CD15 antibody can be a monoclonal antibody. The anti-CD15 antibody can be an IgM antibody or an IgG antibody. The anti-CD15 antibody can be an anti-human CD15 antibody. The concentration of the anti-CD15 antibody can be about 0.001 mg/L to about 15 mg/L. In one embodiment, the concentration of the antibody is 0.05 mg/mL. The concentration of heparin can be between 100 and 100,000 units per liter. A composition further can include divalent cations (e.g., Ca⁺² and/or Mg⁺²).

In another aspect, this document features a composition that includes or consists essentially of dextran; anti-CD15 antibody; heparin; serum albumin; and divalent cations (e.g., Ca⁺² and/or Mg⁺²).

This document also features a kit that includes a blood collection vessel and a cell separation composition described herein. The blood collection vessel can be a blood bag or a vacuum tube.

In another aspect, a method for separating cells is featured. The method includes contacting a blood cell-containing sample with the composition described herein; allowing the sample to partition into an agglutinate and a supernatant phase at 1×g; and recovering the cells from the agglutinate or the supernatant phase. The sample can be a human blood cell-containing sample. The sample can be a peripheral blood sample, an umbilical cord sample, or a bone marrow sample. In some embodiments, cells can be recovered from the supernatant phase. In some embodiments, cells can be recovered from the agglutinate phase.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

This document features compositions and methods for separating cells. As described herein, the compositions specifically aggregate erythrocytes and mature myeloid cells via surface antigen recognition, and stimulate homo- and heterophilic cellular adherence and stimulate the enhanced sedimentation of erythrocytes and myeloid cells at 1×g. Non-erythrocytic, non-myeloid lineage cells, including, for example, leukocytes, stem cells, and progenitor cells can be recovered from the supernatant phase of the fractionated blood sample.

Cell Separation Compositions

A cell separation composition described herein can contain dextran, heparin, serum albumin, and anti-CD 15 antibodies. Dextran is a polysaccharide consisting of glucose units linked predominantly in alpha (1 to 6) mode. Dextran can cause stacking of erythrocytes (i.e., rouleau formation) and thereby facilitate the removal of erythroid cells from solution. Typically, soluble dextran having a molecular weight of 500,000 (e.g., from 400,000 to 550,000, Sigma Chemical Co., St. Louis) is used in compositions described herein.

Cell separation compositions described herein also contain an anticoagulant such as heparin. Heparin can prevent clotting and non-specific cell loss associated with clotting in a high calcium environment. Heparin can be supplied as a heparin salt (e.g., sodium heparin, lithium heparin, or potassium heparin). Typically, the concentration of heparin is between 100 and 100,000 units per liter of composition (e.g., 1,000, 5,000, 10,000, 20,000, 30,000, 40,000, 50,000, 60,000, 70,000, 80,000, or 90,000 units per liter of composition).

A cell separation composition also includes antibodies against (i.e., that have specific binding affinity for) CD15. Anti-CD15 antibodies can cause homotypic agglutination of granulocytes by crosslinking CD15 molecules that are present on the surface of granulocytes. Anti CD15 antibodies also can cause homotypic and heterotypic agglutination of granulocytes with monocytes, NK-cells and B-cells by stimulating expression of adhesion molecules (e.g., L-selectin and beta-2 integrin) on the surface of granulocytes that interact with adhesion molecules on monocytes, NK-cells and B-cells. Heterotypic agglutination of these cell types can facilitate the removal of these cells from solution along with red cell components. Exemplary monoclonal anti-CD15 antibodies include, without limitation, AHN1.1 (Murine IgM Isotype), FMC-10 (Murine IgM Isotype), BU-28 (Murine IgM Isotype), MEM-157 (Murine IgM Isotype), MEM-158 (Murine IgM Isotype), MEM-167 (Murine IgM Isotype). See e.g., Solter D. et al., Proc. Natl. Acad. Sci. USA 75:5565 (1978); Kannagi R. et al., J. Biol. Chem. 257:14865 (1982); Magnani, J. L. et al., Arch Biochem Biophys. 233:501 (1984); Eggens I. et al., J. Biol. Chem. 264:9476 (1989).

Typically, antibodies used in the composition are monoclonal antibodies, which are homogeneous populations of antibodies to a particular epitope contained within an antigen. Suitable monoclonal antibodies are commercially available, or can be prepared using standard hybridoma technology. In particular, monoclonal antibodies can be obtained by techniques that provide for the production of antibody molecules by continuous cell lines in culture, including the technique described by Kohler et al., Nature, 1975, 256:495, the human B-cell hybridoma technique (Kosbor et al., Immunology Today 4:72 (1983); Cole et al., Proc. Natl. Acad. Sci. USA 80:2026 (1983)), and the EBV-hybridoma technique (Cole et al., “Monoclonal Antibodies and Cancer Therapy,” Alan R. Liss, Inc., pp. 77-96 (1983)).

Antibodies can be of any immunoglobulin class including IgG, IgM, IgE, IgA, IgD, and any subclass thereof. Antibodies of the IgG and IgM isotypes are particularly useful in cell separation compositions of the invention. Pentameric IgM antibodies contain more antigen binding sites than IgG antibodies and can be particularly useful for cell separation reagents. Typically, antibodies are provided in a cell separation composition at a concentration between about 0.001 and about 65 mg/L (e.g., between 0.25 to 10, 0.25 to 1, 0.5 to 2, 1 to 2, 4 to 8, 5 to 10, 20 to 40, 42 to 52, or 45 to 65 mg/L). For example, anti-CD15 antibodies can be provided at 0.05 mg/mL.

In some embodiments, a cell separation composition further includes serum albumin (e.g., human or bovine serum albumin) Typically, 0.001 to 1.0 g/L of serum albumin is used. For example, 0.005 to 0.5, 0.0075 to 0.25, 0.01 to 0.02, 0.1 to 0.5, 0.4 to 0.8, or 0.0125 g/L of serum albumin can be used.

Cell separation compositions also can contain divalent cations (e.g., Ca⁺² and/or Mg ⁺²). Divalent cations can be provided, for example, by a balanced salt solution (e.g., Hank's balanced salt solution).

Typically, the composition also contains a buffer (e.g., phosphate buffered saline (PBS)) and has a pH ranging from 6.8 to 7.8 (e.g., 7.4). Other buffers such as MOPS (3-(N-Morpholino) propanesulfonic acid) or HEPES (4-(2-Hydroxyethyl) piperazine-1-ethanesulfonic acid) also can be used.

Compositions described herein can be obtained by combining the components (e.g., dextran, Hank's balanced salt solution, anti-human CD15 antibody, bovine or human serum albumin, and anticoagulant) in water and then stirring the mixture for about 1 to about 30 minutes or until a solution is obtained. For example, 20 g/L dextran, 100 mL/L 10× PBS, 0.05 g/mL anti-human CD15, 0.0125 g/L bovine serum albumin, and 1 mL/L heparin (e.g., 10,000 units/mL sodium heparin) can be combined at room temperature using water to bring the composition to the correct volume and the pH of the composition can be adjusted with sodium hydroxide (e.g., 4N sodium hydroxide).

Methods of Using Cell Separation Compositions

Cells can be separated by contacting a blood cell-containing sample and allowing the sample to partition into an agglutinate and a supernatant phase at 1×g. Cells can be recovered from the supernatant or the agglutinate. The disclosed compositions can be used to separate cells from a variety of blood-cell containing samples, including peripheral blood (e.g., obtained by venipuncture), umbilical cord blood (e.g., obtained post-gravida), and bone marrow (e.g., from aspirate). For example, the compositions described herein can be used to agglutinate erythrocytic cells via surface antigen recognition and mature myeloid cells via stimulated adhesion molecule-mediated cell aggregation.

For example, erythrocytes and mature myeloid cells can be selectively agglutinated using cell separation compositions containing dextran, anti-CD15 antibody, heparin, and serum albumin, allowing non-erythrocytic and non-myeloid cell lineage blood cell components to be recovered from the solution phase (i.e., the supernatant). Thus, agglutinated cells (e.g., erythrocytes and cells of the myeloid lineage) partition away from unagglutinated cells, which remain in solution.

The disclosed compositions and methods can be used to isolate and enrich for a variety of cell types, including, for example, T lymphocytes, T helper cells, T suppressor cells, B cells, hematopoietic stem cells, circulating stem cells (e.g., embryonic or non-embryonic stem cells), circulating fetal cells in maternal circulation, and circulating metastatic tumor cells. The disclosed compositions can be used to agglutinate erythrocytes and myeloid cells of any mammal, including humans, non-human primates, rodents, swine, bovines and equines.

The disclosed compositions can be used, for example, to efficiently prepare cells for tissue culture, immunophenotypic characterization, other diagnostic testing, further purification, and therapeutic administration. The disclosed compositions and methods can be used in the context of allogenic and autologous transplantation.

Cell Separation Kits

A cell separation composition can be combined with packaging material and sold as a kit. The components of a cell separation composition can be packaged individually or in combination with one another. In some embodiments, the packaging material includes a blood collection vessel (e.g., blood bag or vacuum tube). The packaging material included in a kit typically contains instructions or a label describing how the cell separation composition can be used to agglutinate erythrocytes and cells of the myeloid lineage. Components and methods for producing such kits are well known.

The invention is further described in the following examples, which do not limit the scope of the invention described in the claims.

### EXAMPLES

### Example 1

### Cell Separation Reagent

Equal volumes of a cell separation reagent (see Table 1) and a citrate anti-coagulated blood sample are combined in a sealable container (e.g., conical tube or blood collection bag). After mixing, the conical tube or other container is gently mixed on a rocker platform (or by gentle inversion) for 30 to 45 minutes at room temperature. Tubes then are stood upright in a rack for 30 to 50 minutes to permit agglutinated cells to partition away from unagglutinated cells, which remain in solution, and to allow sedimentation. Without disturbing the agglutinate, a pipette is used to recover unagglutinated cells from the supernatant. Recovered cells are washed in phosphate buffered saline (PBS) plus 1% bovine serum albumin or human serum albumin (HSA), or tissue culture medium.

TABLE 1
Cell Separation Composition

| | | |
|---|---|---|
| Dextran | 20 | g/L |
| Hank's buffered saline (10X) | 100 | ml/L |
| Anti-human CD 15 (murine IgM monoclonal antibody; | 0.05 | mg/mL |
| clone 324.B9) | | |
| Bovine Serum Albumin | 0.2 | g/mL |
| Sodium Heparin (10,000 units/ml) | 1 | ml/L |

### Example 2

### Recovery of Leukocytes and Platelets from Normal Adult Bone Marrow

Bone marrow samples were processed using the composition and method of Example 1. Before processing, the number of white blood cells (WBC), red blood cells (RBC), and platelets (PLT) were counted in the samples. Table 2 shows the preprocessing cell count for two bone marrow samples. Table 3 provides the percent recovery of WBC and PLT, percent depletion of RBC, number of adherent cells/mL, number of mesenchymal stem cells (MSC)/mL, and number of days to the culture was confluent. In both samples, MSC were enriched approximately 10,000 fold after processing. MSC were characterized as positive for CD105, CD90, CD73, and negative for CD45, and differentiated to osteoblasts.

TABLE 2
Pre-processing Cell Counts

| | Sample 1 | Sample 2 |
|---|---|---|
| WBC | 277.5 × 10⁶ | 234 × 10⁶ |
| RBC | 52.91 × 10⁹ | 52.065 × 10⁹ |
| PLT | 1720.5 × 10⁶ | 1209 × 10⁶ |

TABLE 3
Post-processing Cell Counts

| | Sample 1 | Sample 2 |
|---|---|---|
| Number of WBC | 58.38 × 10⁶ | 58.1 × 10⁶ |
| WBC Recovery | 21.04% | 24.9% |
| Number of RBC | 0.834 × 10⁹ | 0.83 × 10⁹ |
| RBC Depletion | 98.4% | 98.4 |
| Number of PLT | 1125.9 × 10⁶ | 622.5 × 10⁶ |
| PLT Recovery | 65.4% | 51.5% |
| Adherent cells | 6234/mL | 3910/mL |
| Days to Confluence | 9 | 13 |
| MSC/mL | 758.3 | 100 |

### Other Embodiments

While the invention has been described in conjunction with the foregoing detailed description and examples, the foregoing description and examples are intended to illustrate and not to limit the scope of the invention, which is defined by the scope of the appended claims. Other aspects, advantages, and modifications are within the scope of the claims.

1. A composition comprising:
a) dextran;
b) anti-CD15 antibody;
c) heparin; and
d) serum albumin.

2. The composition of claim 1, further comprising phosphate buffered saline.

3. The composition of claim 1, wherein the pH of said composition is between 6.8 and 7.8.

4. The composition of claim 1, wherein said serum albumin is bovine serum albumin or human serum albumin.

5. (canceled)

6. The composition of claim 1, wherein said anti-CD15 antibody is monoclonal.

7. The composition of claim 1, wherein said anti-CD15 antibody is an IgM antibody or an IgG antibody.

8. The composition of claim 1, wherein said anti-CD15 antibody is an anti-human CD15 antibody.

9. The composition of claim 1, wherein the concentration of said anti-CD15 antibody is about 0.001 mg/L to about 15 mg/L.

10. The composition of claim 1, wherein the concentration of said serum albumin is about 0.5% to about 5%.

11. The composition of claim 1, wherein the concentration of heparin is between 100 and 100,000 units per liter.

12. The composition of claim 1, further comprising divalent cations.

13. The composition of claim 12, wherein said divalent cations are Ca⁺² or

14. (canceled)

15. The composition of claim 12, wherein said divalent cations are Ca⁺² and Mg⁺².

16. The composition of claim 1, wherein the pH of said composition is between 7.2 and 7.4.

17. A composition comprising:
a) dextran;
b) anti-CD15 antibody;
c) heparin;
d) serum albumin; and
e) divalent cations.

18. A kit comprising a blood collection vessel and the cell separation composition of claim 17.

19. The kit of claim 18, wherein said blood collection vessel is a blood bag or a vacuum tube.

20. (canceled)

21. A method for separating cells, said method comprising
a) contacting a blood cell-containing sample with the composition of claim 17;
b) allowing said sample to partition into an agglutinate and a supernatant phase at 1×g; and
c) recovering said cells from said agglutinate or said supernatant phase.

22. The method of claim 21, wherein said sample is a human blood cell-containing sample.

23. The method of claim 21, wherein said sample is a peripheral blood sample, an umbilical cord sample, or a bone marrow sample.

24. (canceled)

25. (canceled)

26. The method of claim 21, wherein said cells are recovered from said supernatant phase.

27. The method of claim 21, wherein said cells are recovered from said agglutinate phase.
